(19) United States
(12) Patent Application Publication
(10) Pub. No.: US 2003/0092599 A1
(43) Pub. Date: May 15, 2003

(54) Fragrance and flavor compositions and fragrance- and flavor-added products

(75) Inventors: Toshikazu Suganuma, Hiratsuka-shi (JP); Keiji Torii, Hiratsuka-shi (JP); Toshio Abe, Hiratsuka-shi (JP); Masakatsu Unno, Hiratsuka-shi (JP); Yasushi Kato, Hiratsuka-shi (JP)

Correspondence Address: SUGHRUE MION, PLLC, 2100 PENNSYLVANIA AVENUE, N.W., WASHINGTON, DC, 20037, US

(73) Assignee: TAKASAGO INTERNATIONAL CORPORATION

(21) Appl. No.: 10/138,559

(22) Filed: May 6, 2002

(30) Foreign Application Priority Data
May 8, 2001 (JP) P.2001-137088

(51) Int. Cl. A61K 7/46

(52) U.S. Cl. 512/1

(57) ABSTRACT

Ketone compounds having marine-like, modern green-like and fatty green-like fragrance and flavor, and fragrance compositions and flavor compositions containing the same. A fragrance or flavor composition which contains one or more of ketone compounds represented by the following general formula (1)

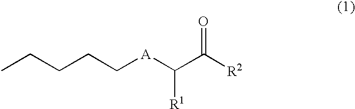

(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group) as the aromatic component.

### FIELD OF THE INVENTION

This invention relates to the field of fragrance and flavor. Illustratively, the invention relates to ketone compounds represented by the following general formula (1)

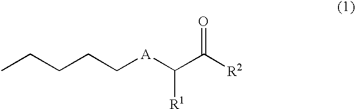

(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group), which have marine-like and modern green-like fragrance characteristics and fatty-like and fatty green-like flavor characteristics, and to use thereof as aromatic components.

### BACKGROUND OF THE INVENTION

With the recent diversification of goods such as various food materials, food additives, food and drink (including table luxuries), aromatic cosmetics, health preservation and sanitation materials, sundries and medicaments, a demand for new fragrance and flavor to be used therein has been increasing and great concern has been directed toward the development of fragrance and flavor materials having unique and highly palatable fragrance and flavor. Particularly with the recent rise in people's intention to love nature, great concern has been directed toward the development of new fragrance and flavor materials derived from natural compounds or those which are similar or analogous to natural compounds, regarding highly palatable marine-like fragrance and flavor, green-like fragrance and flavor and fatty-like fragrance and flavor by which natural environment can be characteristically imagined, and also from the safety point of view.

Great concern has been directed toward the development of fragrance and flavor materials having high palatability, particularly regarding different marine-like and modern green-like fragrance and flavor which are insufficient by the conventionally used marine-like fragrance and flavor including Calone (trade name, Pfizer) and green-like fragrance and flavor including cis-3-hexenol, and fatty-like and milk-like fragrance and flavor which are insufficient by the conventional butter-like fragrance and flavor including acetoin and diacetyl.

### SUMMARY OF THE INVENTION

Accordingly, the object of the invention is to provide ketone compounds having marine-like, modern green-like and fatty green-like fragrance and flavor which satisfy these requirements, and fragrance compositions and flavor compositions containing them.

Under such circumstances, the present inventors have conducted intensive studies with the aim of achieving the object and found as a result of the efforts that high palatability fragrance compositions having modern green-like and marine-like fragrance characteristics and flavor compositions having fatty-like and milk-like flavor characteristics can be obtained by adding ketone compounds represented by the following general formula (1)

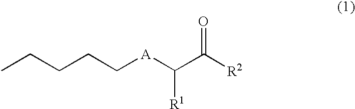

(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group), and further found that these compositions have considerable fragrance and flavor keeping effects. The invention has been accomplished based on these findings.

Accordingly, the invention include each of the following inventions.

(1) A fragrance composition or flavor composition, which comprises one or more of ketone compounds represented by the following general formula (1)

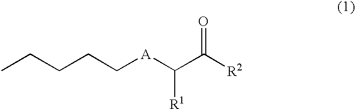

(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group) as the aromatic component.

(2) The fragrance composition or flavor composition described in the item (1), wherein the ketone compounds represented by the general formula (1) are one or more compounds selected from 3-methyl-2,4-nonanedione, 3-methyl-4-hydroxy-2-nonanone, 3,5-decanedione and 5-hydroxy-3-decanone.

(3) The fragrance composition or flavor composition described in the item (1) or (2), wherein one or more of ketone compounds represented by the general formula (1) are used at a concentration of from 0.01 to 50% by weight based on the fragrance composition or from 0.00001 to 5% by weight based on the flavor composition.

(4) A fragrance-added product prepared by adding fragrance with the fragrance composition of any one of the items (1) to (3), wherein it is selected from fragrance products, skin-care cosmetics, make-up cosmetics, hair cosmetics, anti-sunburn cosmetics, medicinal cosmetics, hair-care products, soap, body lotions, bath liquids, detergents, soft finishing agents, cleaning agents, kitchen detergents, bleaching agents, aerosol agents, deodorant-aromatics and sundries.

(5) A product flavored with the flavor composition described in any one of the items (1) to (3), wherein it is selected from flavored food and drink, oral compositions and medicaments.

(6) A method for providing, improving or strengthening fragrance characteristics of a fragrance composition or a fragrance-added product, which comprises adding one or more of ketone compounds represented by the following general formula (1)

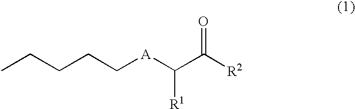

(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group) as the aromatic component.

(7) A method for providing, improving or strengthening flavor characteristics of a flavor composition or a flavored product, which comprises adding one or more of ketone compounds represented by the following general formula (1)

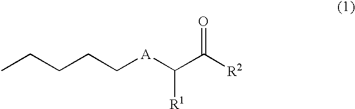

(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group) as the aromatic component.

### DETAILED DESCRIPTION OF THE INVENTION

The following describes the invention further in detail.

The ketone compounds represented by the following general formula (1)

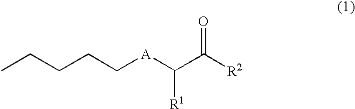

(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group) to be used in the invention are known compounds.

Among these four compounds, three compounds 3,5-decanedione, 3-methyl-4-hydroxy-2-nonanone and 5-hydroxy-3-decanone are substances which have not been studied as fragrance or flavor with no reports on their fragrance and flavor characteristics.

Only 3-methyl-2,4-nonanedione has been reported on its fragrance and flavor characteristics, namely as a lard-like or straw-like odor regarding reversion flavor of soybean [International Patent Publication No. 506,960/1996; FOOD & FOOD INGREDIENTS JOURNAL OF JAPAN, Vol. 168, pp.. 61-76; Fett Wiss. Technol., (1989), 91(6), 225 - 30] and as an aromatic substance of green tea component (Prog. Flavor Precursor Stud. Proc. Inc. Conf., pp. 401 - 407, 1993).

However, nothing has been reported on its use in food flavors as a sea food base flavor or milk base flavor, and nothing has been reported on its use as fragrance.

The ketone compounds of the invention represented by the general formula (1) can be obtained by extracting them from natural materials or by a chemical synthesis method.

In order to obtain the ketone compounds of formula (1) of the invention in a large amount, it is desirable to use a chemical synthesis method.

Production method of the ketone compounds of formula (1) of the invention is carried out, e.g., by the reaction shown below.

Production method of 3-methyl-2,4-nonanedione of the invention is described in the following further in detail.

Production of 3-methyl-2,4-nonanedione is carried out, e.g., by the following method.

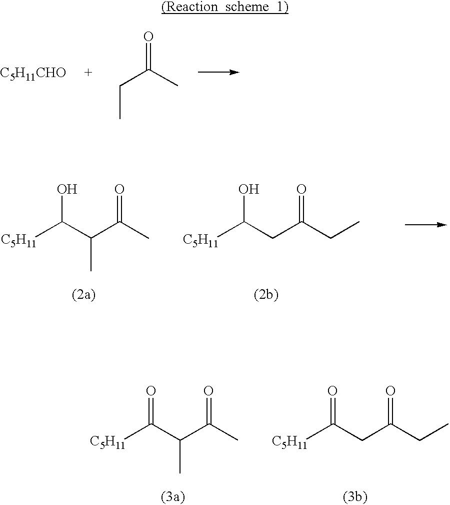

That is, hydroxyketones (2a and 2b) are obtained by carrying out carbon-carbon bond formation reaction of n-hexylaldehyde with methyl ethyl ketone in the presence of a base catalyst and then subjected to oxidation reaction to effect formation of diketones (3a and 3b).

The compound of interest can be isolated by subjecting the thus obtained ketone compounds to a general separation purification method (e.g., a column chromatography).

Illustrative examples of the thus obtained four ketone compounds are 3-methyl-4-hydroxy-2-nonanone (2a), 5-hydroxy-3-decanone (2b), 3-methyl-2,4-nonanedione (3a) and 3,5-decanedione (3b).

As will be shown in the following Examples, all of these compounds have marine-like and modern green-like fragrance characteristics and fatty-like and fatty green-like flavor characteristics, particularly, 3-methyl-4-hydroxy-2-nonanone (2a) and 3-methyl-2,4-nonanedione (3a) have highly palatable excellent marine-like and modern green-like fragrance and odor strength and also have significant fragrance keeping characteristic and stability, and a fragrance composition such as a fragrance providing agent or fragrance improving agent having high palatability can be provided by formulating them.

Also, since 3-methyl-4-hydroxy-2-nonanone (2a) and 3-methyl-2,4-nonanedione (3a) have highly palatable excellent marine-like and modern green-like fragrance characteristics and fatty-like and fatty green-like flavor characteristics, a flavor composition such as a flavor providing agent or flavor improving agent having high palatability can be provided by formulating them.

In addition, the action and effect of desired fragrance keeping and residual fragrance can be particularly increased by formulating the ketone compounds of the invention.

The term “green-like fragrance, marine-like fragrance, modern green-like fragrance, fatty-like flavor and fatty green-like flavor” as used herein means the following fragrance or flavor.

That is, the green-like fragrance is a greenish and refreshing fragrance as if fresh plants or tree leaves are squeezed or a fragrance characteristic to fresh vegetables such as cucumber and white muskmelon, and it means a fragrance which reminds a greenish feeling.

The marine-like fragrance is generally a seashore fragrance generating from the sea and the seashore and means a seaweed-like green fragrance which reminds fragrance of the tide of the sea and the coast.

The modern green-like fragrance means a fresh and young greenish fragrance which, among green-like fragrances, particularly reminds vegetables and un-ripe fruits.

The fatty-like fragrance generally means a fragrance which reminds aroma of oils and fats typified by aliphatic aldehyde compounds, feeling of animal fats such as lard and milk fat such as butter.

In addition, the fatty green-like fragrance generally means which, among green-like fragrances, particularly a greenish fragrance accompanied by a fatty feeling.

A fragrance composition and a flavor composition in which generally used fragrance components are further added to the ketone compounds of the invention can also be used as fragrance components and flavor components. Examples of the other fragrance which can be added include various types of synthetic aroma chemical, natural aroma chemical, natural essential oil, citrus fruit oil and animal aroma chemical, of which floral green base fragrance compositions are particularly desirable, and a broad range of fragrance components described, e.g., in Arctander S., “Perfume and Flavor Chemicals”, published by the author, Montclair, N.J. (U.S.A.), 1969, can be used. Their typical examples include α-pinene, limonene, cis-3-hexenol, phenylethyl alcohol, styralyl acetate, eugenol, rose oxide, linalool, benzaldehyde, muscone and Thesarone (Takasago International Corporation).

Illustratively, when the ketone compounds of the invention are formulated in, e.g., synthetic essential oils such as bergamot oil, galbanum oil, lemon oil, geranium oil, lavender oil and mandarin oil, they can improve effects of the synthetic essential oils by providing mild, full-bodied, fresh and highly palatable fragrance and flavor, as well as their persistency by increasing dispersing and retaining properties, which are originally possessed by natural essential oils. Also, they harmonize well also with citrus fruit essential oils such as of orange, lime and grapefruit and natural essential oils such as lavender oil, vetiver oil, cedar wood oil, citronella oil, geranium oil, lavandine oil and sandal wood oil and can emphasize characteristics of these essential oils, so that they render possible the preparation of novel fragrance composition and flavor composition which are mild, full-bodied and almost natural and also show persistency by increasing dispersing and retaining properties.

In addition, their formulation in flavor compositions such as of strawberry, lemon, orange, grapefruit, apple, pineapple, banana, melon, green tea, Oolong tea, salmon, sardine, small dried sardine, shrimp and crab, which are prepared from , e.g., various types of synthetic aroma chemicals, natural aroma chemicals, natural essential oil, citrus fruit oil and animal aroma chemicals, renders possible preparation of fragrance compositions and flavor compositions which provide mild, full-bodied and almost natural marine-like, modern green-like, fatty-like and fatty green-like aromas, as well as fresh and highly palatable aromas, and also show emphasized persistency by increasing dispersing and retaining properties.

Amount of the ketone compounds of the invention to be formulated in a fragrance composition and a flavor composition varies depending on the kinds of formulating fragrance and flavor and purposes, but in general, the formulating amount is preferably from 0.01 to 50% by weight, particularly from 0.1 to 20% by weight, based on the fragrance composition, or preferably from 0.00001 to 5% by weight, particularly from 0.0001 to 1% by weight, based on the flavor composition.

Also, one or more of other generally used fragrance and flavor retaining agents may be formulated; e.g., agents such as ethylene glycol, propylene glycol, dipropylene glycol, glycerol, hexyl glycol, benzyl benzoate, triethyl citrate, diethyl phthalate, Hercolyn and middle-chain fatty acid triglyceride may be jointly used.

Fragrance-added products and flavored products can be provided by formulating the ketone compounds of the invention alone or a fragrance composition or flavor composition containing the compounds, in an appropriate amount by which the unique fragrance and flavor can be provided, in, e.g., fragrance products, skin-care cosmetics, make-up cosmetics, hair cosmetics, anti-sunburn cosmetics, medicinal cosmetics, hair-care products, soap, body lotions, bath liquids, detergents, soft finishing agents, cleaning agents, kitchen detergents, bleaching agents, aerosol agents, deodorant-aromatics and sundries, or in food and drink, oral compositions and medicaments.

Their illustrative examples include perfumed water, Eau de Parfum, Eau de Toilette and Eau de Cologne as the fragrance products; face washing cream, vanishing cream, cleansing cream, cold cream, massage cream, milky lotion, toilet lotion, beauty wash, pack and make remover as the skin-care cosmetics; foundation, face powder, pressed powder, talcum powder, rouge, lip stick, lip cream, cheek rouge, eye liner, mascara, eye shadow, eyebrow-color, eye pack, nail enamel and enamel remover as the make-up cosmetics; pomade, brillantin, set lotion, hair stick, hair solid, hair oil, hair treatment, hair cream, hair tonic, hair liquid, hair spray, bandlin, hair growth agent and hair dye as the hair cosmetics;

suntan products and sunscreen products as the anti-sunburn cosmetics; antiperspirant, after shaving lotion, gel, permanent wave agent, medicinal soap, medicinal shampoo and medicinal skin cosmetic as the medicinal cosmetics; shampoo, rinse, rinse-in shampoo, conditioner, treatment and hair pack as the hair-care products; toilet soap, bath soap, aromatic soap, transparent soap and synthetic soap as the soap; body soap, body shampoo, shower gel and hand soap as the body lotions; bath agents (e.g., bath salt, bath tablet and bath liquid), form bath (e.g., bubble bath), bath oils (e.g., bath perfume and bath capsule), milk bath, bath jelly and bath cube as the bath liquids;

heavy detergent for clothing use, soft detergent for clothing use, liquid detergent, laundry soap, compact detergent and powder soap as the detergents; softener and furniture care as the soft finishing agents; cleanser, house cleaner, toilette detergent, bath detergent, glass cleaner, fungi remover and drainpipe detergent as the cleaning agents; kitchen soap, kitchen synthetic soap and tableware detergent as the kitchen detergents; oxidation type bleaching agent (e.g., a chlorine base bleaching agent or oxygen base bleaching agent), reduction type bleaching agent (e.g., sulfur base bleaching agent) and optical bleaching agent as the bleaching agents; spray type and powder type aerosols as the aerosol agents; solid type, gel type and liquid type agents as the deodorant-aromatics; and tissue paper and toilette paper as the sundries.

Also, examples of the food and drink include drinks such as fruit juice drinks, fruit wines, milk drinks, carbonated drinks, soft drinks and drinking agents; ices such as ice creams, sherbets and ice candies; favorite drinks such as Japanese- and Western-style confections, jams, candies, jellies, gums, breads, coffees, cocoas, black teas, Oolong teas and green teas; and soups such as a Japanese-style soup, a Western-style soup and a Chinese-style soup, flavor condiments, various convenience drinks and foods and various snack foods; examples of the oral compositions include toothpowder, toothpaste, mouth cleaning agents, mouth washes, troches and chewing gums; and examples of the medicaments include skin external preparations such as adhesive preparations and ointments and oral medicines.

When the ketone compounds of the invention are used in fragrance products, skin-care cosmetics, make-up cosmetics, hair cosmetics, anti-sunburn cosmetics, medicinal cosmetics, hair-care products, soap, body lotions, bath liquids, detergents, soft finishing agents, cleaning agents, kitchen detergents, bleaching agents, aerosol agents, deodorant-aromatics and sundries, or in food and drink, oral compositions and medicaments, they may be used as such or by selecting optional forms depending on each purpose, e.g., in the form of liquid in which they are dissolved in alcohols or polyhydric alcohols such as propylene glycol and glycerol; with natural gummy matters such as gum arabic and tragacanth; in the form of emulsion in which they are emulsified with emulsifying agents such as glycerol fatty acid ester and sucrose fatty acid ester; in the form of powder in which they are coated using natural gummy matters such as gum arabic or fillers such as gelatin and dextrin; in the stabilized or dispersed form in which they are stabilized or dispersed using surface active agents such as a nonionic surface active agent, an anionic surface active agent, a cationic surface active agents and an ampholytic surface active agent; or in the form of microcapsules obtained by treating them with capsule forming agents.

In addition, these fragrance compositions and flavor compositions may be used after making them into stabilized and sustained-release preparations by enclosing them with an including agent such as cyclodextrin. These preparations are used by optionally selecting those which are suited for the final product forms such as liquid, solid, powder gel, mist and aerosol forms.

In this connection, amount of the ketone compounds of the invention to be added to the final products such as fragrance products, skin-care cosmetics, make-up cosmetics, hair cosmetics, anti-sunburn cosmetics, medicinal cosmetics, hair-care products, soap, body lotions, bath liquids, detergents, soft finishing agents, cleaning agents, kitchen detergents, bleaching agents, aerosol agents, deodorant-aromatics and sundries is optionally controlled in response to the expected effects and actions of respective cases and is generally approximately from 0.01 to 25.0% by weight.

Also, amount of the ketone compound (1) to be added to other products such as food and drink, oral compositions and medicaments is optionally controlled in response to the expected effects and actions of respective cases and is generally approximately from 0.0005 to 20% by weight.

The following describes the invention in detail with reference to examples, but the invention is not restricted thereby and the invention is not limited to these examples.

In this connection, the instruments used in the examples for the measurement of physical properties are as follows.

1) Chemical purity

Gas chromatography; Shimadzu GC-14A (mfd. by Shimadzu)

Column; Silicon NB-1 (0.25 mm×30 m) (mfd. by G L Science)

2) Nuclear magnetic resonance spectrum

¹H-NMR; DRX-500 type (500 MHz) (mfd. by Brucker)

Internal standard: tetramethylsilane

3) Infrared spectrum (IR)

Nicolet Avatar 360 FT-IR (mfd. by Nicolet Japan)

4) Mass spectrum (MS)

M-80 Mass Spectrometer: ionization voltage 20 eV (mfd. by Hitachi)

### SYNTHESIS EXAMPLE 1

### Synthesis of 3-methyl-4-hydroxy-2-nonanone (2a) and 5-hydroxy-3-decanone (2b)

In an atmosphere of nitrogen, 10 g (100 mmol) of n-hexyl aldehyde was added dropwise to a mixed solution of 400 mg (10 mmol) of sodium hydroxide, 20 ml of water/methanol (1:1) solution and 28.85 g (400 mmol) of methyl ethyl ketone, at 20 to 25° C. for 8 hours. This was stirred at the same temperature for 15 hours. After completion of the reaction, the organic layer was separated and washed twice with 5% sodium chloride solution and then the organic layer was concentrated under a reduced pressure. The residue was distilled under a reduced pressure to obtain 11.9 g (yield 70%) of a mixture of 3-methyl-4-hydroxy-2-nonanone and 5-hydroxy-3-decanone. It was found by a gas chromatography analysis that the ratio of 3-methyl-4-hydroxy-2-nonanone and 5-hydroxy-3-decanone was 50/50 and their total purity was 90.4%.

By separating and purifying this mixture by a column chromatography using hexane/ethyl acetate as the elution solvent, 5.5 g of 3-methyl-4-hydroxy-2-nonanone and 5.0 g of 5-hydroxy-3-decanone were obtained. Their physical property values are shown below.

<Spectral data of 3-methyl-4-hydroxy-2-nonanone>

Boiling point: 52 - 57° C./0.2 torr. IR (neat): 3446, 2933, 2860, 1705, 1420 cm⁻¹

¹H-NMR (CDCl₃; δ ppm): 0.89 (t), 1.06 (d), 1.07 (d), 1.31 (m), 1.49 (m), 2.19 (s), 2.20 (s), 2.58 (m), 2.62 (m), 3.69 (m), 3.95 (m)

Mass (m/e): 172 (M⁺), 155, 113, 101, 83, 73, 61.

<Spectral data of 5-hydroxy-3-decanone>

¹H-NMR (CDCl₃; δ ppm): 0.87 (t), 1.10 (t), 1.20 - 1.70 (m), 2.00 - 2.45 (m), 3.50 - 3.77 (m)

Mass (m/e): 172 (M⁺), 101, 59.

### SYNTHESIS EXAMPLE 2

### Synthesis of 3-methyl-2,4-nonanedione (3a) and 3,5-decanedione (3b)

In an atmosphere of nitrogen, 10 g (58.1 mmol) of the 3-methyl-4-hydroxy-2-nonanone/5-hydroxy-3-decanone 1:1 mixture obtained in Synthesis Example 1, 50 ml of dichloromethane and 2.41 g (8.7 mmol) of 4-benzoyloxy-2,2,6,6-tetramethylpiperidine-1-oxy were added to an aqueous solution prepared by dissolving 1.17 g (14 mmol) of sodium bicarbonate and 691 mg (5.8 mmol) of potassium bromide in 13.5 ml of water, and the resulting mixture was cooled to 5° C. To this was added dropwise 86.4 ml (174 mmol) of 2.0 mol/liter sodium hypochlorite aqueous solution at 3 to 7° C. for 1.5 hours, and the mixture was further stirred for 30 minutes. After confirming completion of the reaction by a gas chromatography, 1.1 g (8.7 mmol) of sodium sulfite was added thereto, and the organic layer was separated, washed with water, concentrated under a reduced pressure and then roughly distilled under a reduced pressure to obtain 8.42 g of a 1:1 mixture of 3-methyl-2,4-nonanedione and 3,5-decanedione.

By separating and purifying this mixture by a column chromatography using hexane/ethyl acetate as the elution solvent, 4.0 g of 3-methyl-2,4-nonanedione and 3.8 g of 3,5-decanedione were obtained. Their physical property values are shown below.

<Spectral data of 3-methyl-2,4-nonanedione>

Boiling point: 57 - 59° C./0.1 torr. IR (neat): 2957, 2933, 1726, 1702, 1604 cm⁻¹

¹H-NMR (CDCl₃; δ ppm): 0.83 (t), 1.26 (d, J=7.1 Hz), 1.21 (m), 1.28 (m), 1.51 (m), 1.78 (s), 2.06 (s), 2.11 (s), 2.33 (t), 2.41 (m), 3.63 (q, J=7.0 Hz)

Mass (m/e): 170 (M⁺), 99, 71.

<Spectral data of 3,5-decanedione>

IR (neat): 2957, 2933, 1726, 1702, 1604 cm⁻¹

¹H-NMR (CDCl₃; δ ppm): 0.87 (t), 1.10 (t), 1.29 (m), 1.56 (m), 2.28 (m), 5.46 (s), 13.3 (s)

Mass (m/e): 172 (M⁺, 100), 99 (95), 57 (7).

### EXAMPLE 1

### Evaluation of Fragrance and Flavor

Each of the ketone compounds (2a, 2b, 3a and 3b) obtained in Synthesis Examples 1 and 2 was put on a bottle mouth and filter paper, and sensory evaluation was carried out by perfumers having 5 years or more of experience. The evaluation results are shown in Table 1.

TABLE 1

| Compound name | Aroma |
|---|---|
| 3-Methyl-4-hydroxy-2- | Modern green-like and marine-like |
| nonanone (2a) | aroma with a fatty green aroma |
| | having fatty feeling. |
| 5-Hydroxy-3-decanone | Modern green-like and marine-like |
| (2b) | aroma with a fatty green aroma |
| | having fatty feeling. |
| | Weak aroma compared to (2a). |
| 3-Methyl-2,4- | Modern green-like and marine-like |
| nonanedione (3a) | aroma with a fatty green aroma |
| | having fatty feeling. |
| | Similar or weak aroma compared to |
| | (2a). |
| 3,5-Decanedione (3b) | Modern green-like and marine-like |
| | aroma with a fatty green aroma |
| | having fatty feeling. |
| | Weak aroma compared to (3a). |

As is evident from Table 1, all of the compounds were possessed of modern green-like and marine-like aroma with a fatty green aroma having fatty feeling, particularly, 3-methyl-4-hydroxy-2-nonanone (2a) and 3-methyl-2,4-nonanedione (3a) were possessed of highly palatable excellent modern green-like and marine-like aroma and aromatic strength.

### EXAMPLES 2 TO 5

### Fragrance Composition For Floral Fragrance Use

A fragrance composition for floral fragrance use comprised of the following fragrance composition A and the ketone compounds (2a, 2b, 3a and 3b) as active ingredients with their amounts (% by weight) shown in Table 2, having the sum total of 1,000% by weight, was prepared.

### COMPARATIVE EXAMPLES 1 AND 2

As Comparative Examples 1 and 2, a fragrance composition for floral fragrance use comprised of the following fragrance composition A and cis-3-hexenol and dipropylene glycol (to be referred sometimes to as DPG hereinafter) having green-like fragrance with their amounts (% by weight) shown in Table 2, having the sum total of 1,000% by weight, was prepared.

<Fragrance composition A> (% by weight)

| | |
|---|---|
| Benzyl acetate | 270.0 |
| Benzyl salicylate | 137.9 |
| Cinnamyl alcohol | 40.0 |
| Eugenol | 40.0 |
| Galbanum oil | 2.0 |
| 2-Phenylpropanal | 20.0 |
| Indole | 3.0 |
| Kovanol (trade name, mfd. by Takasago | 95.0 |
| International Corporation) | |
| Phenylethyl alcohol | 300.0 |
| Phenylethyl formate | 40.0 |
| Phenylpropyl alcohol | 24.0 |
| Scatol | 0.1 |
| Thesarone (trade name, mfd. by Takasago | 20.0 |
| International Corporation) | |
| Total | 992.0 |

TABLE 2
(% by weight)

Example

| Active ingredient | 2 | 3 | 4 | 5 | 1* | 2* |
|---|---|---|---|---|---|---|
| (2a) | 8 | | | | | |
| (2b) | | 8 | | | | |
| (3a) | | | 8 | | | |
| (3b) | | | | 8 | | |
| cis-3-hexenol | | | | | 8 | |
| DPG | | | | | | 8 |

*= Comparative Example

The fragrance compositions obtained by adding the ketone compounds (2a, 2b, 3a and 3b) of the invention (Examples 2 to 5) were possessed of a floral green tone fragrance with modern green-like and marine-like fragrance, having good balance and high residual fragrance.

On the contrary, the fragrance composition obtained by adding cis-3-hexenol (Comparative Example 1) prominently showed green-like fragrance, thus causing unbalanced floral green tone. Also, the fragrance composition obtained by adding DPG (Comparative Example 2) hardly showed green-like fragrance.

### EXAMPLES 6 TO 9

### Fragrance Composition For Floral Fragrance Use

A fragrance composition for floral fragrance use comprised of the following fragrance composition B and the ketone compounds (2a, 2b, 3a and 3b) as active ingredients with their amounts (% by weight) shown in Table 3, having the sum total of 1,000% by weight, was prepared.

### COMPARATIVE EXAMPLES 3 AND 4

As Comparative Examples 3 and 4, a fragrance composition for floral fragrance use comprised of the following fragrance composition B and cis-3-hexenol and DPG having green-like fragrance with their amounts (% by weight) shown in Table 3, having the sum total of 1,000% by weight, was prepared.

<Fragrance composition B> (% by weight)

| | |
|---|---|
| Ambroxan (trade name, mfd. by Henkel) | 5.0 |
| Benzyl acetate | 15.0 |
| Benzyl salicylate | 200.0 |
| Bergamot oil | 30.0 |
| l-Citronellol (mfd. by Takasago | 15.0 |
| International Corporation) | |
| β-Damascon | 1.0 |
| Dimethylbenzcarbinyl acetate | 10.0 |
| Exaltolide (trade name, mfd. by Firmenich) | 100.0 |
| Hedione (trade name, mfd. by Firmenich) | 100.0 |
| Heliobouquet (trade name, mfd. by Takasago | 60.0 |
| International Corporation) | |
| Iso E Super (trade name, mfd. by IFF) | 100.0 |
| Kovanol (trade name, mfd. by Takasago | 70.0 |
| International Corporation) | |
| δ-Undecalacton | 1.0 |
| Linalool | 30.0 |
| γ-Methyl ionone | 40.0 |
| Oak moss absolute | 3.0 |
| Patchouli oil | 5.0 |
| Phenylethyl alcohol | 100.0 |
| Sandalore (trade name, mfd. by Givaudan) | 70.0 |
| Tonka beans absolute | 20.0 |
| Vanilla resin | 5.0 |
| Total | 980.0 |

TABLE 3
(% by weight)

Example

| Active ingredient | 6 | 7 | 8 | 9 | 3* | 4* |
|---|---|---|---|---|---|---|
| (2a) | 20 | | | | | |
| (2b) | | 20 | | | | |
| (3a) | | | 20 | | | |
| (3b) | | | | 20 | | |
| cis-3-hexenol | | | | | 20 | |
| DPG | | | | | | 20 |

*= Comparative Example

The fragrance compositions obtained by adding the ketone compounds (2a, 2b, 3a and 3b) of the invention (Examples 6 to 9) were possessed of a floral tone fragrance with modern green-like and marine-like fragrance, having good balance and high residual fragrance.

On the contrary, the fragrance composition obtained by adding cis-3-hexenol (Comparative Example 3) prominently showed green-like fragrance, thus causing unbalanced floral bouquet tone. Also, the fragrance composition obtained by adding DPG (Comparative Example 4) hardly showed green-like fragrance.

### EXAMPLES 10 TO 13

### Fragrance Composition For Marine Fragrance Use

A fragrance composition for marine fragrance use comprised of the following fragrance composition C and the ketone compounds (2a, 2b, 3a and 3b) as active ingredients with their amounts (% by weight) shown in Table 4, having the sum total of 1,000% by weight, was prepared.

### COMPARATIVE EXAMPLES 5 AND 6

As Comparative Examples 5 and 6, a marine-like fragrance composition comprised of the following fragrance composition C and Calone (trade name, Pfizer) and DPG having marine-like fragrance with their amounts (% by weight) shown in Table 4, having the sum total of 1,000% by weight, was prepared.

<Fragrance composition C> (% by weight)

| | |
|---|---|
| Orange oil | 200.0 |
| Lavender oil | 100.0 |
| Musk T (trade name, mfd. by Takasago | 150.0 |
| International Corporation) | |
| Benzyl salicylate | 150.0 |
| Hedione (trade name, mfd. by Firmenich) | 100.0 |
| Kovanol (trade name, mfd. by Takasago | 100.0 |
| International Corporation) | |
| Heliobouquet (trade name, mfd. by Takasago | 50.0 |
| International Corporation) | |
| Dehydromyrtenol | 50.0 |
| Woody flor (trade name, mfd. by Takasago | 30.0 |
| International Corporation) | |
| Triplal (trade name, mfd. by IFF) | 20.0 |
| γ-Methyl ionone | 15.0 |
| Eugenol | 10.0 |
| Geranium oil | 5.0 |
| Total | 980.0 |

TABLE 4
(% by weight)

Example

| Active ingredient | 10 | 11 | 12 | 13 | 5* | 6* |
|---|---|---|---|---|---|---|
| (2a) | 20 | | | | | |
| (2b) | | 20 | | | | |
| (3a) | | | 20 | | | |
| (3b) | | | | 20 | | |
| Calone | | | | | 20 | |
| DPG | | | | | | 20 |

*= Comparative Example

The fragrance compositions obtained by adding the ketone compounds (2a, 2b, 3a and 3b) of the invention (Examples 10 to 13) were possessed of a marine tone fragrance with modern green-like fragrance and freshness, having good balance and high residual fragrance.

On the contrary, the fragrance composition obtained by adding Calone (trade name, Pfizer) (Comparative Example 5) prominently showed only a marine tone fragrance and was poor in green feeling, thus spoiling total balance. Also, the fragrance composition obtained by adding dipropylene glycol (Comparative Example 6) hardly showed marine-like and modern green-like fragrance.

### EXAMPLES 14 TO 17

### Fruit Flavor Composition

A fruit flavor composition comprised of the following flavor composition D and the ketone compounds (2a, 2b, 3a and 3b) as active ingredients with their amounts (% by weight) shown in Table 5, having the sum total of 100% by weight, was prepared.

### COMPARATIVE EXAMPLES 7 AND 8

As Comparative Examples 7 and 8, a fruit flavor composition comprised of the following flavor composition D and cis-3-hexenol having green-like flavor and propylene glycol (to be referred sometimes to as PG hereinafter) with their amounts (% by weight) shown in Table 5, having the sum total of 100% by weight, was prepared.

<Flavor composition D>

| | |
|---|---|
| Isoamyl acetate | 2.0 |
| Isobutyl butyrate | 12.0 |
| Ethyl acetate | 8.0 |
| Ethyl levulinate | 25.0 |
| Ethyl butyrate | 5.0 |
| Hexyl butyrate | 2.0 |
| Hexanol | 1.0 |
| Cis-3-Hexenol | 20.0 |
| Methyl anthranilate | 4.0 |
| 2-Methylbutyric acid | 1.0 |
| trans-2-Hexenyl acetate | 1.0 |
| Ethyl propionate | 1.0 |
| Linalool | 1.0 |
| Styralyl acetate | 4.0 |
| trans-2-Hexenyl butyrate | 1.0 |
| Propylene glycol | 11.9 |
| Total | 99.9 |

TABLE 5
(% by weight)

Example

| Active ingredient | 14 | 15 | 16 | 17 | 7* | 8* |
|---|---|---|---|---|---|---|
| (2a) | 0.1 | | | | | |
| (2b) | | 0.1 | | | | |
| (3a) | | | 0.1 | | | |
| (3b) | | | | 0.1 | | |
| cis-3-hexenol | | | | | 0.1 | |
| PG | | | | | | 0.1 |

*= Comparative Example

The flavor compositions obtained by adding the ketone compounds (2a, 2b, 3a and 3b) of the invention (Examples 14 to 17) were possessed of a fruit-like flavor with marine-like and fatty green-like flavor, having good balance and high residual flavor.

On the contrary, the flavor composition obtained by adding cis-3-hexenol (Comparative Example 7) prominently showed green-like flavor, thus causing unbalanced fruit-like flavor. Also, the flavor composition obtained by adding PG (Comparative Example 8) hardly showed marine-like and fatty green-like flavor.

### EXAMPLES 18 TO 21

### Milk Flavor Composition

A milk flavor composition comprised of the following flavor composition E and the ketone compounds (2a, 2b, 3a and 3b) as active ingredients with their amounts (% by weight) shown in Table 6, having the sum total of 990% by weight, was prepared.

### COMPARATIVE EXAMPLES 9 AND 10

As Comparative Examples 9 and 10, a milk flavor composition comprised of the following fragrance composition E and acetoin having a fatty-like flavor and PG with their amounts (% by weight) shown in Table 6, having the sum total of 990% by weight, was prepared.

<Flavor composition E>

| | |
|---|---|
| Dimethyl sulfide | 0.1 |
| Octanal | 0.2 |
| 2-Undecanone | 0.2 |
| 2-Nonanone | 0.3 |
| Ethyl cyclothene | 0.4 |
| Ethyl levulinate | 0.8 |
| Ethyl maltol | 1.0 |
| Butyric acid | 2.0 |
| Caproic acid | 2.0 |
| Caprylic acid | 3.0 |
| Vanillin | 3.0 |
| Butylbutyryl lactate | 5.0 |
| Capric acid | 6.0 |
| δ-Decalactone | 18.0 |
| δ-Undecalactone | 20.0 |
| δ-Decalactone | 23.0 |
| Propylene glycol | 904.9 |
| Total | 989.9 |

TABLE 6
(% by weight)

Example

| Active ingredient | 18 | 19 | 20 | 21 | 9* | 10* |
|---|---|---|---|---|---|---|
| (2a) | 0.1 | | | | | |
| (2b) | | 0.1 | | | | |
| (3a) | | | 0.1 | | | |
| (3b) | | | | 0.1 | | |
| acetoin | | | | | 0.1 | |
| PG | | | | | | 0.1 |

*= Comparative Example

The flavor compositions obtained by adding the ketone compounds (2a, 2b, 3a and 3b) of the invention (Examples 18 to 21) were possessed of a milk-like flavor with marine-like and fatty green-like flavor, having good balance and high residual flavor.

On the contrary, the flavor composition obtained by adding acetoin (Comparative Example 9) prominently showed a butter-like flavor, thus causing unbalanced milk-like flavor. Also, the flavor composition obtained by adding propylene glycol (Comparative Example 10) hardly showed marine-like and fatty green-like flavor.

### EXAMPLES 22 TO 25

### Crab Flavor Composition

A crab flavor composition comprised of the following flavor composition F and the ketone compounds (2a, 2b, 3a and 3b) as active ingredients with their amounts (% by weight) shown in Table 7, having the sum total of 1,000% by weight, was prepared.

### COMPARATIVE EXAMPLES 11 AND 12

As Comparative Examples 11 and 12, a crab flavor composition comprised of the following flavor composition F and Undaria extract having fatty-like flavor and PG with their amounts (% by weight) shown in Table 7, having the sum total of 990% by weight, was prepared.

<Flavor composition F>

| | |
|---|---|
| Indole 0.1% | 0.1 |
| Methional 1% | 0.1 |
| Diacetyl 1% | 1.0 |
| Lauric acid 1% | 1.5 |
| Capric acid 1% | 2.0 |
| Fusel oil | 0.1 |
| Ethyl laurate | 0.1 |
| Ethyl levulinate | 0.2 |
| 2,6-Nonadienal 1% | 3.0 |
| Hexadecanal | 0.2 |
| Acetic acid 1% | 5.0 |
| Sulfurol | 0.3 |
| Dimethyl sulfide | 0.4 |
| Crab extract | 70.0 |
| Propylene glycol | 905.9 |
| Total | 989.9 |

TABLE 7
(% by weight)

Example

| Active ingredient | 22 | 23 | 24 | 25 | 11* | 12* |
|---|---|---|---|---|---|---|
| (2a) | 0.1 | | | | | |
| (2b) | | 0.1 | | | | |
| (3a) | | | 0.1 | | | |
| (3b) | | | | 0.1 | | |
| Undaria extract | | | | | 0.1 | |
| PG | | | | | | 0.1 |

*= Comparative Example

The flavor compositions obtained by adding the ketone compounds (2a, 2b, 3a and 3b) of the invention (Examples 22 to 25) were possessed of a crab-like flavor with marine-like and fatty green-like flavor, having good balance and high residual flavor.

On the contrary, the flavor composition obtained by adding Undaria extract (Comparative Example 11) prominently showed a seaweed-like flavor but smelled fishy, thus causing unbalanced crab-like flavor. Also, the flavor composition obtained by adding propylene glycol (Comparative Example 12) hardly showed marine-like and fatty green-like flavor.

### EXAMPLES 26 TO 48

The fragrance compositions and flavor compositions of the invention were used to prepare a cosmetic cream (Example 26), a lotion (Example 27), a milky lotion (Example 28), a sunscreen cream (Example 29), a hair tonic (Example 30), a shampoo composition (Example 31), a rinse composition (Example 32), a body shampoo composition (Example 33), a bath agent composition (Example 34), a powder detergent composition (Example 35), a liquid detergent composition (Example 36), a tableware detergent composition (Example 37), a softener composition (Example 38), a soap composition (Example 39), an antiperspirant composition (Example 40), an aromatic composition (Example 41), a milk coffee composition (Example 42), a seafood flavor composition (Example 43), mouth wash composition (Example 44), a toothpaste composition (Example 45), a troche composition (Example 46), a chewing gum composition (Example 47) and a candy composition (Example 48).

When use feeling of these preparations was evaluated, all of them showed excellent palatability and good use feeling.

### EXAMPLE 26

### Formulation Example (Cosmetic Cream)

A cosmetic cream was prepared using the fragrance composition for floral fragrance use prepared in Example 2.

<Cosmetic cream> (% by weight)

| | |
|---|---|
| Stearyl alcohol | 6.0 |
| Stearic acid | 2.0 |
| Hydrogenated lanolin | 4.0 |
| Squalane | 9.0 |
| Octyl decanol | 10.0 |
| Glycerol | 6.0 |
| Polyethylene glycol 1500 | 4.0 |
| Polyoxyethylene (25) cetyl ether | 3.0 |
| Glycerol monostearate | 2.0 |
| Methylparaben | adequate amount |
| Ethylparaben | adequate amount |
| Fragrance composition of Example 2 | 0.1 |
| Purified water | balance |
| Total | 100.0 |

### EXAMPLE 27

### Formulation Example (Lotion)

A lotion was prepared using the fragrance composition for floral fragrance use prepared in Example 6.

<Lotion> (% by weight)

| | |
|---|---|
| Sorbitol | 2.0 |
| Concentrated glycerol | 2.0 |
| 1,3-Butylene glycol | 2.0 |
| Polyethylene glycol 1000 | 1.0 |
| Polyethylene oleyl ether (25 E.O.) | 2.0 |
| Ethanol | 10.0 |
| Methylparaben | 0.02 |
| Fragrance composition of Example 6 | 0.01 |
| Purified water | balance |
| Total | 100.0 |

### EXAMPLE 28

### Formulation Example (Milky Lotion)

A milky lotion was prepared using the fragrance composition for floral fragrance use prepared in Example 6.

<Milky lotion> (% by weight)

| | |
|---|---|
| Squalane | 3.0 |
| Vaselin | 1.0 |
| Stearyl alcohol | 0.3 |
| Sorbitan monostearate | 1.5 |
| Polyoxyethylene (20) sorbitan monostearate | 3.0 |
| 1,3-Butylene glycol | 5.0 |
| Fragrance composition of Example 6 | 0.1 |
| Purified water | balance |
| Total | 100.0 |

### EXAMPLE 29

### Formulation Example (Sunscreen Cream)

The components for solution A use shown below were uniformly mixed while heating at 80 to 85° C. Also, the components for solution B use shown below were uniformly mixed while heating at 75° C. The solution A, while stirring, was gradually added to the solution B to carry out emulsification, and the emulsion was further stirred using a homogenizer, cooled to 50° C., mixed with a component C (the fragrance composition for marine fragrance use prepared in Example 10), cooled to 30° C. and then packed in a container to prepare a sunscreen cream.

<Sunscreen cream>

| <Solution A> | |
|---|---|
| Parsol 1789 (mfd. by Givaudan) | 1.0 |
| Spermaceti wax | 8.0 |
| Glyceryl tricaprylate | 12.0 |
| Cetyl alcohol | 2.0 |
| Stearyl alcohol | 2.0 |
| Methylparaben | 0.15 |
| <Solution B> | |
| Glycerol | 1.5 |
| Propylene glycol | 1.5 |
| Purified water | 68.6 |
| Sodium p-hydroxybenzoate | 0.15 |
| <Component C> | |
| Fragrance composition of Example 10 | 0.1 |
| Total | 97.00 |

### EXAMPLE 30

### Formulation Example (Hair Tonic)

A hair tonic was prepared using the fragrance composition for marine fragrance use prepared in Example 10.

<Hair tonic> (% by weight)

| | |
|---|---|
| Ethanol | 50.0 |
| Ethyl oleate | 1.0 |
| Polyoxyethylene (40) hydrogenated castor oil | 2.0 |
| Fragrance composition of Example 10 | 0.1 |
| Purified water | balance |
| Total | 100.0 |

### EXAMPLE 31

### Formulation Example (Shampoo Composition)

A shampoo composition was prepared using the fragrance composition for floral fragrance use prepared in Example 6, by stirring the following components at 80° C. until they became uniform and then cooling the mixture to 35° C.

<Shampoo composition> (% by weight)

| | |
|---|---|
| Sodium lauryl sulfate | 40.00 |
| N-Coconut oil fatty acid acyl-N-carboxymethoxyethyl- | 10.00 |
| N-carboxtmethylethylenediamine disodium | |
| Coconut oil fatty acid diethanolamide (2) | 2.00 |
| Butylene glycol | 2.00 |
| Citric acid | 0.35 |
| Sodium chloride | 0.10 |
| Methylparaben | 0.20 |
| Propylparaben | 0.10 |
| Tetrasodium edetate | 0.10 |
| Fragrance composition of Example 6 | 0.50 |
| Purified water | balance |
| Total | 100.00 |

### EXAMPLE 32

### Formulation Example (Rinse Composition)

A rinse composition was prepared using the fragrance composition for floral fragrance use prepared in Example 2.

<Rinse composition> (% by weight)

| | |
|---|---|
| O-[2-Hydroxy-3-(trimethylammoniopropyl] | 0.10 |
| hydroxyethylcellulose chloride | |
| Sodium hydroxide | 0.03 |
| Citric acid | 0.05 |
| Methylparaben | 0.20 |
| Propylparaben | 0.10 |
| Polyoxyethylene cetyl ether (10 E.O.) | 0.50 |
| Cetanol | 1.50 |
| Behenyl alcohol | 3.00 |
| Distearyldimethylammonium chloride (90%) | 0.20 |
| Stearyltrimethylammonium chloride (50%) | 1.50 |
| Cetyl 2-ethylhexanoate | 0.50 |
| Methyl polysiloxane | 2.00 |
| Fragrance composition of Example 2 | 0.50 |
| Purified water | balance |
| Total | 100.00 |

### EXAMPLE 33

### Formulation Example (Body Shampoo Composition)

A body shampoo composition was prepared using the fragrance composition for floral fragrance use prepared in Example 4.

<Body shampoo composition> (% by weight)

| | |
|---|---|
| Dibutylhydroxytoluene | 0.05 |
| Methylparaben | 0.10 |
| Propylparaben | 0.10 |
| Tetrasodium edetate | 0.10 |
| Potassium chloride | 0.20 |
| Glycerol | 5.00 |
| Coconut oil fatty acid diethanolamide (2) | 3.00 |
| Polyoxyethylene lauryl ether sodium acetate | 10.00 |
| (3 E.O.) (30%) | |
| Coconut oil fatty acid amide propylbetaine | 25.00 |
| Solution (34%) | |
| Potassium myristate (40%) | 25.00 |
| Fragrance composition of Example 4 | 0.50 |
| Purified water | balance |
| Total | 100.00 |

### EXAMPLE 34

### Formulation Example (Bath Agent Composition)

A bath agent composition was prepared using the fragrance composition for marine fragrance use prepared in Example 12.

<Bath agent composition> (% by weight)

| | |
|---|---|
| Sodium chloride | 10.0 |
| Potassium chloride | 6.0 |
| Sodium bicarbonate | 33.0 |
| Borax | 2.0 |
| Glycerol | 0.2 |
| Silicic anhydride | 1.0 |
| 1,3-Butylene glycol | 0.001 |
| Plant extract powder (Matricaria | 2.0 |
| chamomilla, Angelica acutiloba) | |
| Fragrance composition of Example 12 | 0.5 |
| Dry sodium sulfate | 45.299 |
| Total | 100.0 |

### EXAMPLE 35

### Formulation Example (Powder Detergent Composition)

A powder detergent composition was prepared using the fragrance composition for floral fragrance use prepared in Example 2.

<Powder detergent composition> (% by weight)

| | |
|---|---|
| Sodium linear alkylbenzenesulfonate | 15.6 |
| Sodium carbonate | 30.0 |
| Sodium silicate | 6.3 |
| Carboxymethylcellulose | 0.5 |
| Zeolite | 20.0 |
| Sodium percarbonate | 11.3 |
| Tetraacetylethylenediamine | 2.5 |
| Silicon defoaming agent | 3.2 |
| Fragrance composition of Example 2 | 0.2 |
| Purified water | balance |
| Total | 100.0 |

### EXAMPLE 36

### Formulation Example (Liquid Detergent Composition)

A liquid detergent composition was prepared using the floral fragrance composition prepared in Example 6.

<Liquid detergent composition> (% by weight)

| | |
|---|---|
| C13 Alcohol EO (9) | 55.0 |
| Diethanolamine | 10.0 |
| m-Xylenesulfonic acid diethanolamine salt | 2.0 |
| Enzyme | 0.6 |
| Hexylene glycol | 15.0 |
| Fluorescent | 0.1 |
| Fragrance composition of Example 6 | 0.2 |
| Purified water | balance |
| Total | 100.0 |

### EXAMPLE 37

### Formulation Example (Tableware Detergent Composition)

A tableware detergent composition was prepared using the fragrance composition for floral fragrance use prepared in Example 3.

<Tableware detergent composition> (% by weight)

| | |
|---|---|
| Alkyl (C12) glucoside | 15.0 |
| Sodium polyoxyethylene (3) dodecyl ether sulfate | 10.0 |
| 1,3-Dipropylene glycol | 1.5 |
| Ethanol | 6.5 |
| Fragrance composition of Example 3 | 0.1 |
| Purified water | balance |
| Total | 100.0 |

### EXAMPLE 38

### Formulation Example (Softener Composition)

A softener composition was prepared using the fragrance composition for floral fragrance use prepared in Example 6.

<Softener composition> (% by weight)

| | |
|---|---|
| Dialkyldimethylammonium chloride | 15.0 |
| Polyoxyethylene (30) lauryl ether | 3.0 |
| Fatty acids | 1.0 |
| Dimethyl polysiloxane | 0.5 |
| Ethylene glycol | 5.0 |
| Methylparaben | 0.1 |
| Tetrasodium edetate | 0.1 |
| Fragrance composition of Example 6 | 0.5 |
| Purified water | balance |
| Total | 100.0 |

### EXAMPLE 39

### Formulation Example (Soap Composition)

A soap composition was prepared using the fragrance composition for marine fragrance use prepared in Example 12.

<Soap composition>

| | |
|---|---|
| Lauric acid monoglyceride sulfuric acid | 55.0 |
| ester sodium salt | |
| Lauryl sulfate ester sodium salt | 10.0 |
| Soap | 30.0 |
| Cetyl alcohol | 4.0 |
| Titanium dioxide | 1.0 |
| BHT | adequate amount |
| Dyestuff | adequate amount |
| Tetrasodium edetate | adequate amount |
| Fragrance composition of Example 12 | 1.0 |

### EXAMPLE 40

### Formulation Example (Antiperspirant Composition)

An antiperspirant composition was prepared using the fragrance composition for floral fragrance use prepared in Example 2.

<Antiperspirant composition> (% by weight)

| | |
|---|---|
| Aluminum chlorohydrate | 10.0 |
| Anhydrous ethyl alcohol | 60.0 |
| 1,3-Butylene glycol | 3.0 |
| Benzalkonium chloride | 0.2 |
| Polyoxyethylene (40) hydrogenated | 0.5 |
| castor oil | |
| Water-soluble thickener | 1.0 |
| Fragrance composition of Example 2 | 0.5 |
| Purified water | balance |
| Total | 100.0 |

### EXAMPLE 41

### Formulation Example (Oily Gel Aromatic Composition)

An oily gel aromatic composition was prepared using the fragrance composition for marine fragrance use prepared in Example 10.

<Oily gel aromatic composition> (% by weight)

| | |
|---|---|
| Sodium stearate | 7.5 |
| Purified water | 2.0 |
| Hexylene glycol | 4.0 |
| Dibutylhydroxytoluene | 0.2 |
| d-Limonene | 76.3 |
| Fragrance composition of Example 10 | 2.0 |
| Purified water | balance |
| Total | 100.0 |

### EXAMPLE 42

### Formulation Example (Milk Coffee Composition)

A milk coffee composition was prepared using the milk flavor composition prepared in Example 18.

<Mi1k coffee composition>

| Coffee extract | 19.0 | g |
|---|---|---|
| Regular coffee | 60.0 | g |
| Instant coffee | 1.0 | g |
| Granulated sugar | 75.0 | g |
| Whole milk powder | 6.5 | g |
| Skim milk powder | 6.0 | g |
| Sugar ester | 0.5 | g |
| Sodium bicarbonate | 0.7 | g |
| | (balance) | |
| Flavor composition of Example 18 | 1.0 | ml |
| Coffee flavor | 1.0 | ml |
| Purified water | balance | |
| Total | 1000.0 | g |

### EXAMPLE 43

### Formulation Example (Seafood Flavor Composition)

A seafood composition was prepared using the crab flavor composition prepared in Example 22.

<Seafood composition> (% by weight)

| | |
|---|---|
| Raw fish meat paste | 500.0 |
| Sodium chloride | 14.0 |
| Sweet sake for seasoning | 19.0 |
| Albumen | 39.0 |
| Potato starch | 34.0 |
| Corn starch | 30.0 |
| Sodium glutamate | 5.0 |
| 5′-Ribonucleotide 2 Na | 0.5 |
| Purified sugar | 1.5 |
| Yeast extract powder | 0.5 |
| Flavor composition of Example 18 | 5.0 |
| Crab extract | 3.5 |
| Cool water | 348.0 |
| Total | 1000.0 |

### EXAMPLE 44

### Formulation Example (Mouth Wash Composition)

(1) Preparation of mouth wash flavor composition

A mouth wash flavor composition was prepared using the fruit flavor composition prepared in Example 14.

<Mouth wash flavor composition> (% by weight)

| | |
|---|---|
| l-Menthol | 50.0 |
| Peppermint oil top cut | 20.0 |
| Eucalyptus oil | 10.0 |
| Flavor composition of Example 14 | 10.0 |
| Anethole | 6.0 |
| Sage oil | 2.0 |
| Eugenol | 1.0 |
| Fennel oil | 0.8 |
| Thyme oil | 0.2 |
| Total | 100.0 |

(2) Preparation of mouth wash composition

A mouth wash composition was prepared using the mouth wash flavor composition prepared in the above (1).

<Mouth wash composition> (% by weight)

| | |
|---|---|
| 95% Ethyl alcohol | 15.00 |
| 70% Sorbitol solution | 10.00 |
| Polyoxyethylene hydrogenated castor oil (EO 60) | 2.00 |
| Mouth wash flavor composition of (1) | 0.10 |
| Sodium benzoate | 0.05 |
| Saccharin sodium | 0.02 |
| Purified water | balance |
| Total | 100.00 |

### EXAMPLE 45

### Formulation Example (Toothpaste Composition)

(1) Preparation of toothpaste flavor composition

A toothpaste flavor composition was prepared using the fruit flavor composition prepared in Example 14.

<Toothpaste flavor composition> (% by weight)

| | |
|---|---|
| Peppermint oil | 35.0 |
| l-Menthol | 25.0 |
| Spearmint oil | 10.0 |
| Flavor composition of Example 14 | 10.0 |
| Anethole | 8.0 |
| Sweet orange oil | 5.0 |
| Clove oil | 5.0 |
| Lemon oil | 2.0 |
| Total | 100.0 |

(2) Preparation of toothpaste composition

A toothpaste composition was prepared using the toothpaste flavor composition prepared in the above (1).

<Toothpaste composition> (% by weight)

| | |
|---|---|
| Calcium hydrogenphosphate | 50.00 |
| (calcium secondary phosphate) | |
| Glycerol | 24.00 |
| 3-l-Menthoxypropane-1,2-diol | 0.10 |
| Carboxymethylcellulose sodium | 1.50 |
| Sodium lauryl sulfate | 1.40 |
| Toothpaste flavor composition of (1) | 1.00 |
| Saccharin sodium | 0.15 |
| Xylitol | 0.15 |
| Sodium benzoate | 0.05 |
| Purified water | balance |
| Total | 100.00 |

### EXAMPLE 46

### Formulation Example (Troche Composition)

(1) Preparation of oral fresh flavor composition

An oral fresh flavor composition was prepared using the fruit flavor composition prepared in Example 16.

<Oral fresh flavor composition> (% by weight)

| | |
|---|---|
| l-Menthol | 50.0 |
| Lemon oil | 15.0 |
| Peppermint oil | 10.0 |
| 1,8-Cineole | 5.0 |
| Lime oil | 5.0 |
| Flavor composition of Example 18 | 5.0 |
| Ethyl alcohol | 10.0 |
| Total | 100.0 |

(2) Preparation of troche composition

A troche composition was prepared using the oral fresh flavor composition prepared in the above (1).

<Troche composition> (% by weight)

| | |
|---|---|
| 95% Ethyl alcohol | 50.0 |
| Glycerol | 10.0 |
| Polyoxyethylene hydrogenated castor oil (EO 60) | 2.0 |
| Oral fresh flavor composition of (1) | 1.5 |
| Sorbitol | 0.2 |
| Xylitol | 0.1 |
| Purified water | balance |
| Total | 100.0 |

### EXAMPLE 47

### Formulation Example (Chewing Gum Composition)

(1) Preparation of flavor composition for chewing gum use

A flavor composition for chewing gum use was prepared using the fruit flavor composition prepared in Example 14.

<Composition for chewing gum use> (% by weight)

| | |
|---|---|
| Flavor composition of Example 14 | 5.0 |
| Peppermint oil | 44.5 |
| Spearmint oil | 10.0 |
| l-Menthol | 5.0 |
| Methyl salicylate | 5.0 |
| Eucalyptus oil | 10.0 |
| Clove oil | 0.5 |
| Total | 100.0 |

(2) Preparation of chewing gum composition

A chewing gum composition was prepared using the flavor composition for chewing gum use prepared in the above (1).

<Chewing gum composition> (% by weight)

| | |
|---|---|
| Gum base | 20 |
| Powdered sugar | 66 |
| Starch syrup | 13 |
| Flavor composition for chewing gum use of (1) | 1 |
| Total | 100 |

### EXAMPLE 48

### Formulation Example (Black Tea Flavor Composition For Candy Use)

(1) Preparation of black tea flavor composition for candy use

A black tea flavor composition for candy use was prepared using the milk flavor composition prepared in Example 18.

<Black tea flavor composition for candy use> (% by weight)

| | |
|---|---|
| Flavor composition of Example 18 | 5.0 |
| Linalool | 0.1 |
| Linalool oxide | 0.02 |
| Citral | 0.05 |
| α-Terpineol | 0.002 |
| cis-3-Hexenol | 0.05 |
| trans-3-Hexenol | 0.002 |
| Methyl salicylate | 0.001 |
| Black tea extract | 50.0 |
| Ethyl alcohol | 44.775 |
| Total | 100.0 |

(2) Preparation of black tea candy composition

A black tea candy composition was prepared using the black tea flavor composition for candy use prepared in the above (1).

<Black tea candy composition>

| | | |
|---|---|---|
| Granulated sugar | 540.0 | g |
| Starch syrup | 480.0 | g |
| Purified water | 160.0 | g |
| Plant hydrogenated oil | 20.0 | g |
| Lecithin | 0.2 | g |
| Flavor composition for candy use of (1) | 0.8 | g |

By adding the ketone compounds of the invention, highly palatable fragrance compositions having modern green-like and marine-like fragrance characteristics and flavor compositions having fatty-like and milk-like flavor characteristics can be obtained. These compositions can be used in a broad range of applications such as fragrance and flavor or various food materials, food additives, food and drink, aromatic cosmetics and health preservation and sanitation materials, which contain the ketone compounds of the invention as the active ingredients.

While the invention has been described in detail and with reference to specific embodiments thereof, it will be apparent to one skilled in the art that various changes and modifications can be made therein without departing from the scope thereof.

This application is based on Japanese patent application No. 2001-137088 filed May 8, 2001, the entire contents thereof being hereby incorporated by reference.

What is claimed is:

1. A fragrance composition which comprises one or more of ketone compounds represented by the following general formula (1) 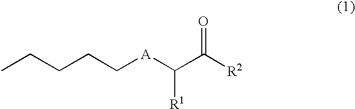
(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group) as the aromatic component.

2. The fragrance composition according to claim 1, wherein the ketone compounds represented by the general formula (1) are one or more compounds selected from 3-methyl-2,4-nonanedione, 3-methyl-4-hydroxy-2-nonanone, 3,5-decanedione and 5-hydroxy-3-decanone.

3. The fragrance composition according to claim 1, wherein one or more of ketone compounds represented by the general formula (1) are used at a concentration of from 0.01 to 50% by weight based on the fragrance composition.

4. The fragrance composition according to claim 2, wherein one or more of ketone compounds represented by the general formula (1) are used at a concentration of from 0.01 to 50% by weight based on the fragrance composition.

5. A fragrance-added product prepared by adding fragrance with the fragrance composition of any one of claims 1 to 4, wherein it is selected from fragrance products, skin-care cosmetics, make-up cosmetics, hair cosmetics, anti-sunburn cosmetics, medicinal cosmetics, hair-care products, soap, body lotions, bath liquids, detergents, soft finishing agents, cleaning agents, kitchen detergents, bleaching agents, aerosol agents, deodorant-aromatics and sundries.

6. A method for providing, improving or strengthening fragrance characteristics of a fragrance composition or a fragrance-added product, which comprises adding one or more of ketone compounds represented by the following general formula (1) 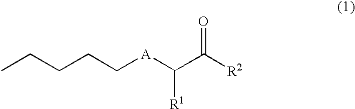
(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group) as the aromatic component.

7. A flavor composition which comprises one or more of ketone compounds represented by the following general formula (1) 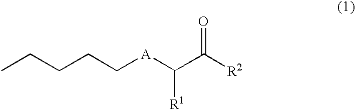
(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group) as the aromatic component.

8. The flavor composition according to claim 7, wherein one or more of ketone compounds represented by the general formula (1) are used at a concentration of from 0.00001 to 5% by weight based on the flavor composition.

9. A product flavored with the flavor composition of claim 8.

10. The flavored product according to claim 9, wherein it is a product selected from food and drink, oral compositions and medicaments.

11. A method for providing, improving or strengthening flavor characteristics of a flavor composition or a flavored product, which comprises adding one or more of ketone compounds represented by the following general formula (1) 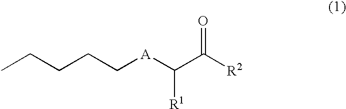
(wherein A is C═O or CH—OH, R¹ is hydrogen atom or methyl group and R² is methyl group or ethyl group, with the proviso that R² is ethyl group when R¹ is hydrogen atom, and R² is methyl group when R¹ is methyl group) as the aromatic component.
